(19) United States
(12) Patent Application Publication
(10) Pub. No.: US 2002/0153021 A1
(43) Pub. Date: Oct. 24, 2002

(54) Washing and sterilizing line and uses thereof

(75) Inventor: Etienne Audet, Sainte-Foy (CA)

Correspondence Address: OGILVY RENAULT, 1981 MCGILL COLLEGE AVENUE, SUITE 1600, MONTREAL, QC, H3A2Y3, CA

(73) Assignee: CFR ASSAINISSEMENT INC., Sainte-Claire (CA)

(21) Appl. No.: 10/107,351

(22) Filed: Mar. 28, 2002

Related U.S. Application Data
(60) Provisional application No. 60/279,755, filed on Mar. 30, 2001.

(51) Int. Cl. B08B 3/12

(52) U.S. Cl. 134/1; 134/30; 134/10; 134/111; 134/67; 134/184

(57) ABSTRACT

The present invention relates to a washing system and method designed for automatically wash, dry and sterilize items. The items may consists in racks, utensils, dish. Items are submitted first to a washing process, than are conveyed through a drying-sterilizing zone.

### BACKGROUND OF THE INVENTION

1. Field of the Invention

The invention relates to dishwashing assembly, and more particularly, to assembly of different cleaning apparatuses specifically adapted for carrying out a method of washing, sterilizing and drying commercially utilized items.

2. Description of Prior Art

Dishwashers fall into two generally distinct but somewhat overlapping categories, namely, domestic or home machines, and commercial machines such as are used in restaurants, institutions or other public facilities. This latter category is itself divisible into various types of commercial machines.

Two of the most common types of commercial machines are the single rack-type and the conveyor-type. The former includes a single chamber into which a rack of soiled ware can be placed. Within the chamber, the entire cleaning process including washing, rinsing and drying is performed on the rack. Multiple racks must be washed sequentially, with each rack being completely cleaned before the next can be operated upon. A conveyor-type machine, on the other hand, includes a conveyor for carrying individual items or entire racks of ware through multiple stations within the machine A different operation is carried out at each station, such as washing, rinsing, or drying. Thus, multiple items or racks of ware can be placed on the conveyor and moved continuously through the machine so that, for example, while one item or rack is being rinsed, a preceding item or rack can be dried.

Examples of both of these types of commercial dishwashing machines can be found in U.S. Pat. No. 4,439,242 issued Mar. 27, 1984 to Hadden.

Dishwashers generally, and conveyor-type dishwashers in particular, are relatively energy intensive in their operation. For example, substantial quantities of water heated to at least temperatures of 120° C. are introduced into the machine at the final rinse stage.

Moreover, significant energy is required to drive the two or more pump motors, which are typically rated in the range of one horsepower. A relatively large motor is also required to drive the conveyor, and electrical and steam heaters are required to maintain washing liquid at an adequate temperature within the machine tanks. A heater and blower motor are required for the drying operation, and various other components requiring energy input are present throughout the machine. Consequently, it is of great economic advantage to a machine owner for energy usage within the machine to be kept as low as possible.

One of the problems in making conveyor-type dishwasher energy efficient results from the varying patterns of use that are typical with such machines. During peak periods, such as at meal times, a large quantity of dishware will typically pass through the machine. Therefore, it is not unusual at such times for ware items or racks to pass through the machine at closely spaced intervals, so that the machine is essentially completely full for extended periods of time. In such a case, there is little waste of energy in having each component at each station of the machine in simultaneous operation.

Many continuous-flow types of dishwashing machines have been developed for use by industrial, institutional, and the other like concerns for washing dishes and other utensils in a continuous manner. Such dishwashing machines usually include a washing compartment containing washing devices for washing the dishes and the other utensils passing therethrough, and a closed loop conveyor for conveying the utensils through the washing compartment. In the conventional dishwashing apparatus of this type, the conveyor is made of elements permanently or semi-permanently connected in a closed loop arranged in two tiers, including an upper horizontal stretch which receives the utensils for conveying them through the washing compartment, and a lower return stretch.

Traditional dishwasher systems and methods of this general type present a variety of limitations and disadvantages which are particularly apparent in commercial or institutional kitchens wherein large quantifies of different ware items must be washed on a frequent or near-continuous basis. In such facilities, manual dishwasher techniques involving manual handling and scrubbing of each ware item in a hot water detergent bath are generally being replaced in favor of dishwasher appliances designed to speed the washing process while reducing manual labor requirements and labor costs associated therewith. However, it is widely recognized that many types of food debris such as dried-on or baked-on material are extremely difficult or impossible to remove without individual manual scrubbing of the ware item. Accordingly, while automated dishwasher appliances have provided significant improvements in faster washing with lower labor costs, most commercial and institutional kitchens utilize a pre-rinse station whereat soiled kitchen ware items are manually inspected one at a time, and if appropriate, heavy or stubborn debris is removed with a combination of jetted hot water, detergent, and/or manual scrubbing. As a result, substantial manual labor and related labor costs are still required. Moreover, the requirement for hot water at pre-rinse and wash stations results in high system energy usage. Still further, this requisite manual handling of the individual ware items in a generally wet and slippery environment increases the risk of breakage to fragile glass ware and the like, as well as risk of employee injury upon exposure to broken glass items and/or to the generally wet and slippery work environment.

In addition, increased attention has focused in recent years upon the relatively high usage rate of water and detergent in commercial dishwashing systems inclusive of pre-rinse stations. That is, such dishwashing systems can require several hundred thousand gallons of water each year in a single restaurant establishment, together, with a corresponding usage of several thousand gallons of detergent. This high water use can be extremely undesirable in geographical areas having a limited water supply due to drought or general arid conditions. Moreover, the resultant high rate of discharge of water and chemicals to the local sewage or septic system, particularly in conjunction with large quantities of removed food material, can be extremely undesirable.

Alternative cleaning methods utilizing ultrasound principles are known in the art wherein one or more ultrasonic signal generators provide cavitation bubbles within a water bath for vigorously scrubbing a substrate to be cleaned. More specifically, the ultrasonic generator radiates a high frequency sound wave through the water bath, with the rapidly alternating wave producing a sequence of localized positive and negative pressure regions. The negative pressure regions effectively separate or cavitate the water bath to produce a large quantity of minute bubbles. As these cavitation bubbles within the water bath are contacted against a surface to be cleaned, the bubbles implode and thus provide a substantial mechanical scrubbing action, which can be effective in dislodging and removing debris from a submerged target substrate.

In institutional kitchens, such as hotel or restaurant kitchens, large quantities of dishes are washed daily. Placing the dishes in racks, which are conveyed through a washing line, effects washing. Such washing lines usually comprise pre-wash, main wash and rinsing.

The operating costs of a washing line consist of water, energy and detergent consumed. Previously attention has not been paid to what the idle running costs of such a washing line may be. Unexpectedly, calculations show that the idle running costs of a kitchen washing e.g. 500 standard-sized racks daily may, at worst, exceed several dollars.

It would be highly desirable to be provided with a new economic and environmentally compatible method and apparatus for continuously wash, sterilize and dry, domestic and industrial utensils, dishes or racks, on a line that can be used on one place or being used directly by being fixed on a plate-form with which the apparatus can be displaced from one site to another one.

### SUMMARY OF THE INVENTION

One object of the present invention is to provide new economic and environmentally compatible method and apparatus for continuously wash, sterilize and dry, domestic and industrial utensils, dishes or racks, on a line that can be used on one place or being used directly by being fixed on a trailer, a transportable plate-form or on any vehicle with which the apparatus can be displaced from one site to another one.

Another object of the present invention is to provide an apparatus and a method for washing and drying-sterilizing items having the result that fewer particles adhere to the substrate.

Another object of the present invention is to provide an apparatus and a method for washing and drying-sterilizing items having the result that adhesion of particles to the substrate caused by static electricity in particular can be reduced.

Still another object of the present invention is to provide an apparatus and a method for rinsing and drying a substrate having the result that adhesion of particles to the substrate in a rinsing solution in particular can be reduced.

A further object of the present invention is to provide an apparatus and a method for washing and drying-sterilizing items having the result that adhesion of particles to the substrate can be reduced, and in which the substrate can be dried quickly.

In accordance with the present invention there is provided a ware item washing system, comprising;

a washing sub-zone including a bath comprising washing solution for receiving ware item to be cleaned; and at least one ultrasound generator submerged within the washing solution; a transducer for controlling the ultrasound generator; a filter system for filtering the washing solution for eliminating particles from the washing solution and recycling a portion of filtered washing solution in the washing sub-zone; and

a closed drying-sterilizing sub-zone for drying and sterilizing the items washed in the washing sub-zone, comprising at least on conveyor for conveying the item through the drying-sterilizing sub-zone; at least one air diffuser system to create air displacement in the drying-sterilizing sub-zone; at least one ultraviolet, ion, or chemical emitting source functioning alone or in combination for sterilizing item during passage through the drying-sterilizing sub-zone.

The washing sub-zone may comprises an ozone generator ozonizing said washing solution, and the drying-sterilizing sub-zone may comprise a washing solution recovery plate.

Another object of the present invention is to provide a method for washing and sterilizing items one an automatic washing line comprising the steps of;

a) contacting item to be cleaned with a washing solution in a bath comprising at least one ultrasound generator;

b) submitting the washed items of step a) to air-drying combined to at least one of ultraviolet radiation, ionized air, or chemical agent.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates a side elevation of parts of the washing-sterilizing line according to one embodiment of the present invention;

FIG. 2 illustrates a top plan of the washing-sterilizing line according to one embodiment of the present invention;

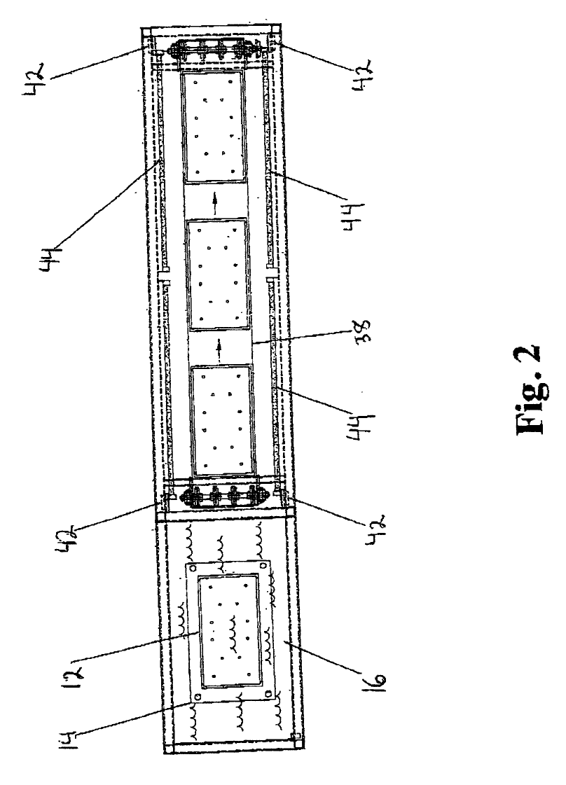

FIG. 3 illustrates an end elevation of the washing-sterilizing line according to one embodiment of the present invention;

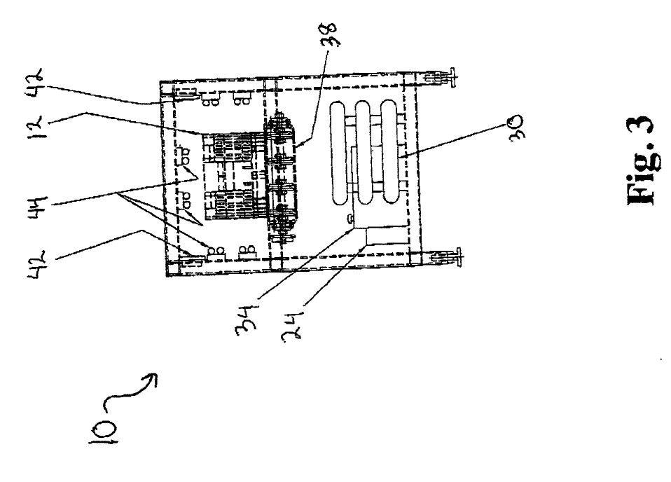

FIG. 4 illustrates an side elevation of an external view of the washing-sterilizing line according to one embodiment of the present invention;

FIG. 5 illustrates a top plan of an external view of the washing-sterilizing line according to one embodiment of the present invention; and

FIG. 6 illustrates a side elevation of parts of the washing-sterilizing line according to a second embodiment of the present invention.

It will be noted that throughout the appended drawings, like features are identified by like reference numerals.

### DETAILED DESCRIPTION OF THE INVENTION

In accordance with the present invention, there is provided a washing-sterilizing line composed of a washing sub-zone 18 and a drying-sterilizing sub-zone 36

The dishwashing apparatus illustrated in the drawings is particularly useful as an industrial or institutional washing machine requiring large volumes of dishes or other utensils to be washed in a continuous manner. The illustrated apparatus comprises a washing line, generally designated 10, containing the washing sub-zone 18, for washing the utensils passing therethrough, and a drying-sterilizing sub-zone, generally designated 36, for drying and sterilizing the utensils or racks after they have been washed in the washing sub-zone. The utensils or racks are conveyed through sub-zones 18 and 36 by an immersion plate 14, and conveyer, generally designated 38.

As mentioned, the washing line 10 is divided in two sub-zones. The first one, the washing sub-zone 18 comprise an immersion plate 14, performing soaking of rack 12, into a bath 16 in which there is a washing solution; an ultrasound generator 26 emits ultrasounds closely regulated by a transducer 24 connected to a control switch 20; and a liquid level sensor 28. The washing solution may be heated with heater 22, and be replaced on a continuous way depending of needs, or recovered by circulating through a filter system 30, where waste and debris are eliminated from the washing solution before being returned to the bath 16. The washing solution may be replaced at a very low rate in this manner by new solution provided by from a tank 34. The washing solution may also be ozonized when passing through an ozone generator 32.

The drying-sterilizing sub-zone 36, comprises a conveyor 38, which perform passage of racks 12 through the drying-sterilizing sub-zone 36; during passage of racks 12 in the sub-zone 36, the racks 12 are submitted to air pulsed by fans 42, and ultraviolet radiations emitted by ultra-violet emitting sources 44. The air may be ionized or ozonized prior being pulsed in the drying-sterilizing sub-zone 36, and ozone level controlled by an ozone level sensor 50.

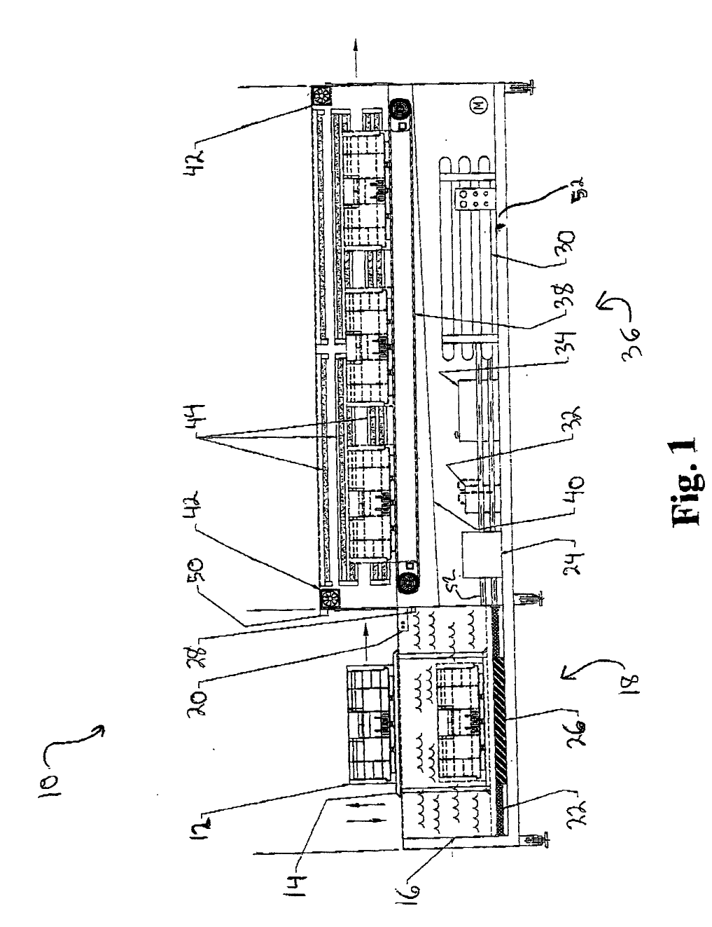

Referring to FIG. 1, in one embodiment of the invention, the washing method operates with a washing line 10 comprising a step at the beginning of the line where are placed racks 12. A tray may be used in place of the rack. The racks 12 or trays may contain items to be washed and sterilized, the racks 12 being automatically displaced on an immersion plate 14 that plunge items in a washing bath 16 containing washing solution for which the level is regulated by a liquid level sensor 28. Alternatively, other types of vertical transport systems such as inclined conveyor segments and the like may be used. The washing solution which may be alternatively pre-ionized, and ozonized by passing through an ozone generator 32, and microfiltrated through a filtration system 30. At least one ultrasonic generator 26 is mounted on the interior walls of the bath 16, with a preferred arrangement being shown in FIG. 1 to include one generator 28 mounted at the bottom wall of the bath.

Each of the ultrasonic signal generators has a construction known in the art to include an ultrasonic transducer element mounted onto a resonant plate or similar structure. When energized, the transducer elements oscillate the resonant plate to produce a high pitched and high frequency ultrasonic wave that is radiated to the surrounding environment. In the dishwasher system 10, the transducer elements 24 and resonant plates 26 are contained within sealed casings immersed within the bath 16, such that the ultrasonic signals are radiated to the bath 16.

The ultrasonic signal imparted to the water bath 16 creates rapidly fluctuating positive and negative pressure variations at localized zones within the water bath as the ultrasonic signal travels therethrough. The negative pressure causes the liquid to pull apart and thereby create tiny or minute cavitation bubbles. Activation of the ultrasound generators 26 thus produces large quantities of virtually millions of such cavitation bubbles which pass through the water bath and contact the surfaces of the ware items 12 to be cleaned with an implosive action. This implosive action of the cavitation bubbles provides a vigorous and highly effective cleaning action for removing food debris and the like from the surfaces of the ware items. This cleaning action is enhanced in preferred forms of the invention by heating the water bath 16 to a temperature in the range of about 40 to 95° C., and by incorporating a minor proportion of a cleaning product in the washing solution in the bath. A preferred cleaning product comprises a non-foaming or low foaming and biodegradable surfactant, and added to the washing solution.

The cleaning product provided by the ultrasonic generators 26 is closely regulated by the transducer-controller 24 to prevent damage to or breakage of fragile kitchenware items. The controller 24 variably selects the specific frequency of the ultrasonic signal emanating from the generators 26. In this regard, the frequency of the generated signal is rapidly altered to prevent the occurrence of standing or resonant waves within the water bath. Although the specific range of ultrasonic frequencies can vary, as well as the specific frequency variation function, a contemplated signal frequency range is on the order of 25-60 kilohertz. The sweep time for cycling through the range of ultrasonic frequencies occurs rapidly with each cycle within about 0.01-1.00 second.

After a certain time in the washing sub-zone 18, racks 12 are then automatically displaced in and conveyed on a conveyor 38, through a drying-sterilizing sub-zone 36 that may contain a combination of ionized air and ozone diffused by fans 42.

In one embodiment of the present invention, ULPA (ultra-low particulate air) filtered air drying with an ionization air system for static elimination may be provided. Also provided are an ultraviolet light source installed in the drying-sterilizing sub-zone 36 to insure complete exposure of any resist contamination, and spray delivery of high normality universal resist developer. These features provide for complete and rapid removal of resist contamination using a chemical that does not require special waste handling. The system can be effective for cleaning any resist-contaminated part used in items washing.

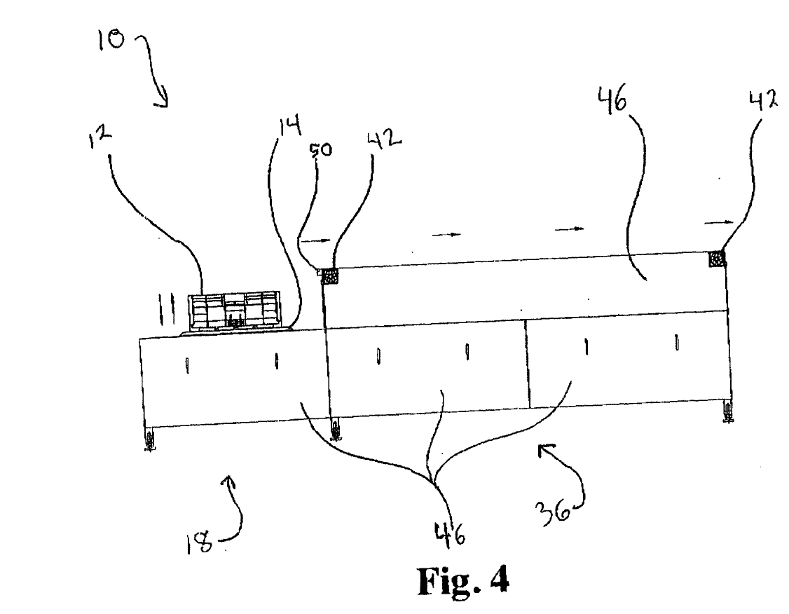

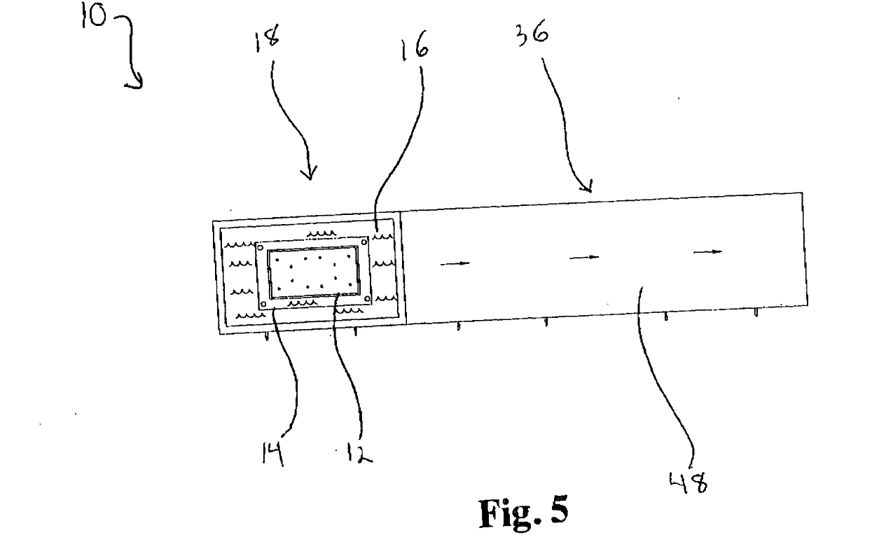

Referring to FIGS. 4 and 5, washing 18 and drying-sterilizing 36 sub-zones are covered with side-panels 46 and top panels 48.

It will be understood that the air is heated to improve its drying capability, is filtered to minimize the likelihood of introducing foreign particles into the chamber 10, and is ionized to minimize any charge picked up by the air by virtue of its movement. This in turn minimizes the likelihood of the cleaned parts having electrostatic charges, which may attract particles out of the air.

According to another embodiment of the present invention, there may be provided a combination of ionized air and ozone that may allow in the same time in processus for the transformation of water on items into hydrogen peroxide (H₂O₂).

In another embodiment of the invention, there is provided a washing line that can be used in institutional kitchens, such as hotel or restaurant kitchens, or in any unit or department where is performed a step of washing and sterilization of food or pharmaceutical containers and dishes. The invention is used where is washed and sterilized daily large quantities of dishes and food containers. Washing is effected by placing the items in racks that are conveyed through a washing line. Such washing lines usually comprise a washing zone with a cleaning composition, followed by a sterilization-drying zone with ionized air and ozone.

In one embodiment of the invention, the washing apparatus of the invention may be transportable from one place to another one by being fixed on a trailer or any useful vehicle. Such a setting may allow performing the washing method of the present invention temporally and in a place where there is a need, and where there is limited reserve of water.

In accordance with further aspects of the invention, the washing solution constituting the bath 16 can be filtered and recycled to provide substantial savings in water usage. In particular, FIG. 1 shows the filter circuit to include a recirculated conduit 52 through which the water bath 16 can be drawn for passage through a filtration system 30 prior to return circulation to the washing sub-zone 18. The filtration system 30 is designed to remove entrained food material and the like from the washing solution, thereby advantageously permitting such material to be separately collected and disposed while additionally permitting washing solution re-use. The filtration step may proceed continuously during system operation, or intermittently between cleaning sequences as a basket of ware items is submerged within the water bath. The washing solution, if necessary, may be composed of water only.

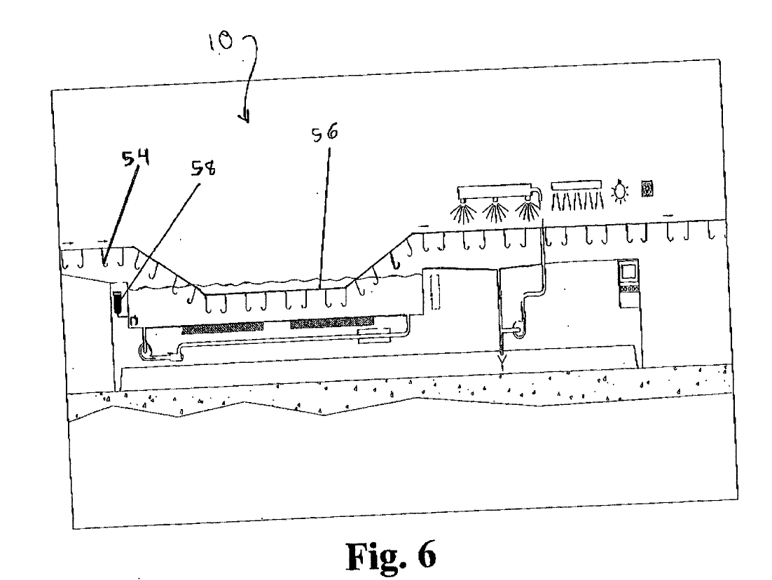

One skilled in the art will contemplate a second embodiment of the present invention as shown in FIG. 6. As this second embodiment is the same as the previously described embodiment, only little differences will be pointed out. According to the second embodiment of the present invention, articles or items to be washed and sterilized are displaced through the washing-sterilizing line 10 with carrier systems 54 secured on a securing mean 56 consisting in an endless chain or wire cable, rather than on a rack.

A dispenser 58 can be placed in manner to dispense according to a determined flow a washing concentrate in the washing solution. The washing concentrate can be any chemical or product acting as a soap like product in the cleaning process of items to be washed and sterilized. The washing concentrate may comprise also different enzymes well known in the art and utilized to wash dishes for example.

The washing process according to the invention has a small duration, substantially shorter to that of a conventional single-phase washing process, and therefore requires amounts of water, detergent and energy which are substantially also lower with respect to the conventional washing process.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

1. A ware item washing system, comprising:
a washing sub-zone including a bath comprising washing solution for receiving ware item to be cleaned; and at least one ultrasound generator submerged within said washing solution; a transducer for controlling said ultrasound generator; a filter system for filtering said washing solution for eliminating particles from said washing solution and recycling a portion of filtered washing solution in said washing sub-zone; and
a closed drying-sterilizing sub-zone for drying and sterilizing said items washed in said washing sub-zone, comprising at least on conveyor for conveying said item through said drying-sterilizing sub-zone; at least one air diffuser system to create air displacement in the drying-sterilizing sub-zone; at least one ultraviolet, ion, or chemical emitting source functioning alone or in combination for sterilizing item during passage through said drying-sterilizing sub-zone.

2. The washing system of claim 1, wherein said washing sub-zone comprises an ozone generator ozonizing said washing solution.

3. The washing system of claim 1, wherein said drying-sterilizing sub-zone comprises a washing solution recovery plate.

4. A method for washing and sterilizing items one an automatic washing line comprising the steps of;
b) contacting item to be cleaned with a washing solution in a bath comprising at least one ultrasound generator;
b) submitting said washed items of step a) to air-drying combined to at least one of ultraviolet radiation, ionized air, or chemical agent.

5. A method of claim 5, wherein said washing solution is ozonized.

6. A method of claim 5, wherein said washing solution is filtered-recirculated during a washing cycle.

7. A method of claim 5, wherein air in step of air-drying is further ionized during said air-drying.
